(19) United States
(12) Patent Application Publication
(10) Pub. No.: US 2025/0180454 A1
(43) Pub. Date: Jun. 5, 2025

(54) METHOD AND APPARATUS FOR ADAPTING OPERATION SETTINGS FOR A FREQUENCY SWEEP MEASUREMENT OF A POLYMER PRODUCT

(71) Applicant: BOREALIS AG, Vienna (AT)

(72) Inventors: Vasileios Touloupidis, Linz (AT); Bernhard Knogler, Linz (AT); Vitor Barroso, Linz (AT); Anna-Karin Jarl, Stenungsund (SE); Christoph Kaufmann, Burghausen (DE); Niclas Carlsson, Stenungsung (SE); Staffan Skalen, Stenungsund (SE)

(21) Appl. No.: 18/834,510

(22) PCT Filed: Feb. 13, 2023
(86) PCT No.: PCT/EP2023/053435

§ 371 Date: Jul. 30, 2024

(30) Foreign Application Priority Data
Feb. 14, 2022 (EP) 22 156 581.5

(51) Int. Cl. G01N 11/00; G01N 33/44

(52) U.S. Cl. CPC G01N 11/00; G01N 33/442

(57) ABSTRACT

Method for adapting operation settings for a frequency sweep measurement of a polymer product, the method comprising the steps of: providing (S1) at least one set of operation setting parameters comprising at least one frequency range for the frequency sweep measurement of the polymer product, the at least one frequency range comprising a plurality of specific measurement frequencies; optimizing (S2) the frequency range by deleting at least one measurement frequency of the plurality of specific measurement frequencies within a lower half of the frequency range resulting in a first optimized frequency range; and optimizing (S3) the first optimized frequency range by determining a value of measurement frequencies present per decade of frequency within at least a subrange of the frequency range and reducing the determined value of measurement frequencies present per decade within the at least one subrange of the frequency range by deleting at least one measurement frequency of the at least one subrange resulting in a second optimized frequency range.

### FIELD OF THE INVENTION

The present invention relates to methods for polymer rheological characterization via fast frequency sweep and in particular to a method and an apparatus for adapting operation settings for a frequency sweep measurement of a polymer product.

### BACKGROUND OF THE INVENTION

In order to monitor and control the properties of a polymer during production, reactor sample needs to be collected and characterized with a material- or parameter-characterizing method of choice at regular intervals, defining the manipulative moves.

The measurement feedback frequency, for classic common-use reactors typically every 4 to 6 hours, limits the selection of the characterization methods only to those that can provide results within this timeframe. For instance, the total measurement time includes sample acquisition, sample preparation, measurement preparation, sample heating-up, and actual measurement. Furthermore, additional limitations connected to technical expertise level and plant resources further apply.

Ideally, the knowledge of the actual molecular weight distribution would be needed as it is the basis of all secondary properties (rheological, mechanical). In practice however, this is prohibitive for full-scale plant environment (high level resources needed in terms of expertise personnel, materials and high measurement time).

Thus, there is a need to improve polymer rheological characterization as used in current polymer reactors.

### SUMMARY OF THE INVENTION

The foregoing and other objects are solved by the subject-matter of the present invention as defined by the independent claims. Further embodiments are defined by the dependent claims.

According to a first aspect of the present invention, a method is provided, a method for adapting operation settings for a frequency sweep measurement of a polymer product.

The method comprises the step of providing at least one set of operation setting parameters comprising at least one frequency range for the frequency sweep measurement of the polymer product, the at least one frequency range comprising a plurality of specific measurement frequencies.

The method comprises the step of optimizing the frequency range by deleting at least one measurement frequency of the plurality of specific measurement frequencies within a lower half of the frequency range resulting in a first optimized frequency range.

The method comprises the step of optimizing the first optimized frequency range by determining a value of measurement frequencies present per decade of frequency within at least a subrange of the frequency range and reducing the determined value of measurement frequencies present per decade within the at least one subrange of the frequency range by deleting at least one measurement frequency of the at least one subrange resulting in a second optimized frequency range.

In other words, the present invention advantageously provides a set of special operation settings for the frequency sweep measurement that leads to significant reduction of measurement time while the method accuracy remains at an adequate level for the needs of plant control.

Further, the present invention advantageously allows that the settings lead to significant reduction of the measurement time.

According to one exemplary embodiment of the present invention, after the step of optimizing the frequency range by deleting at least one measurement frequency, the first optimized frequency range is further optimized by equally distributing the remaining specific measurement frequencies within the lower half of the frequency range or within the complete frequency range.

According to one exemplary embodiment of the present invention, after the step of optimizing the first optimized frequency range, the second optimized frequency range is further optimized by equally distributing the remaining specific measurement frequencies within the subrange of the frequency range or within the complete frequency range.

According to one exemplary embodiment of the present invention, the method further comprises the step of validating a consistency of the second optimized frequency range by comparing a calculated complex viscosity value to a measured melt flow index value using the second optimized frequency range.

According to one exemplary embodiment of the present invention, the method further comprises the steps of applying the second optimized frequency range for quality control measurements of the polymer product.

According to one exemplary embodiment of the present invention, the method further comprises the steps of applying the second optimized frequency range for a currently employed melt flow index measurement. This step might be combined with the previously mentioned step of applying the second optimized frequency range for quality control measurements of the polymer product.

According to one exemplary embodiment of the present invention, the method further comprises the steps of generating the second optimized frequency range for a specific polymer grade of interest of the polymer product.

According to one exemplary embodiment of the present invention, by generating the second optimized frequency range for the specific polymer grade of interest of the polymer product, a frequency sweep fingerprint for the specific polymer grade is defined.

According to one exemplary embodiment of the present invention, the set of operation setting parameters comprises at least one shear rate.

According to a second aspect of the present invention, a computer program product is provided comprising computer-readable instructions which, when loaded and executed on processor, performs the method according to any one of the embodiments of the first aspect or the first aspect as such.

According to a third aspect of the present invention, an apparatus is provided, the apparatus configured for adapting operation settings for a frequency sweep measurement of a polymer product, the apparatus comprising a data memory and a processor.

The data memory is configured to provide at least one set of operation setting parameters comprising at least one frequency range for the frequency sweep measurement of the polymer product, the at least one frequency range comprising a plurality of specific measurement frequencies.

The processor is configured to optimize the frequency range by deleting at least one measurement frequency of the plurality of specific measurement frequencies within a lower half of the frequency range resulting in a first optimized frequency range.

The processor is configured to optimize the first optimized frequency range by determining a value of measurement frequencies present per decade of frequency within at least a subrange of the frequency range and reducing the determined value of measurement frequencies present per decade within the at least one subrange of the frequency range by deleting at least one measurement frequency of the at least one subrange resulting in a second optimized frequency range.

According to one embodiment of the present invention, the processor is further configured to optimize the first optimized frequency range by equally distributing the remaining specific measurement frequencies within the lower half of the frequency range or within the complete frequency range.

According to one embodiment of the present invention, the processor is further configured to optimize the second optimized frequency range by equally distributing the remaining specific measurement frequencies within the subrange of the frequency range or within the complete frequency range.

According to one embodiment of the present invention, the processor is further configured apply the second optimized frequency range for quality control measurements of the polymer product.

According to one embodiment of the present invention, the processor is further configured to apply the second optimized frequency range for a currently employed melt flow index measurement.

A computer program performing the method of the present invention may be stored on a computer-readable medium. A computer-readable medium may be a floppy disk, a hard disk, a CD, a DVD, an USB (Universal Serial Bus) storage device, a RAM (Random Access Memory), a ROM (Read Only Memory) and an EPROM (Erasable Programmable Read Only Memory).

A computer-readable medium may also be a data communication network, for example the Internet, which allows downloading a program code, with a connection via WLAN or 3G/4G or any other wireless data technology.

The methods, systems and devices described herein may be implemented as software in a Digital Signal Processor, DSP, in a micro-controller or in any other side-processor or as hardware circuit within an application specific integrated circuit, ASIC, CPLD or FPGA.

The present invention can be implemented in digital electronic circuitry, or in computer hardware, firmware, software, or in combinations thereof, e.g. in available hardware of conventional mobile devices or in new hardware dedicated for processing the methods described herein.

### BRIEF DESCRIPTION OF THE DRAWINGS

A more complete appreciation of the invention and the attendant advantages thereof will be more clearly understood by reference to the following schematic drawings, which are not to scale, wherein:

FIG. 1 shows a schematic diagram of a method for adapting operation settings for a frequency sweep measurement of a polymer product according to an exemplary embodiment of the invention; and

FIG. 2 shows a schematic diagram of an apparatus for adapting operation settings for a frequency sweep measurement of a polymer product according to an exemplary embodiment of the invention.

### DETAILED DESCRIPTION OF EMBODIMENTS

The illustration in the drawings is schematically and not to scale. In different drawings, similar or identical elements are provided with the same reference numerals.

Generally, identical parts, units, entities or steps are provided with the same reference symbols in the figures.

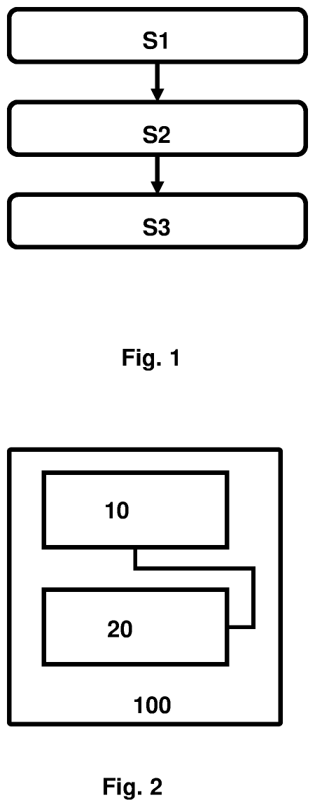

FIG. 1 shows a schematic diagram of a method for adapting operation settings for a frequency sweep measurement of a polymer product according to an exemplary embodiment of the invention.

In particular, FIG. 1 shows a method for adapting operation settings for a frequency sweep measurement of a polymer product, the method comprising the following steps.

As a first step of the method, providing S1 at least one set of operation setting parameters comprising at least one frequency range for the frequency sweep measurement of the polymer product, the at least one frequency range comprising a plurality of specific measurement frequencies is conducted.

As a second step of the method, optimizing S2 the frequency range by deleting at least one measurement frequency of the plurality of specific measurement frequencies within a lower half of the frequency range resulting in a first optimized frequency range is performed.

As a third step of the method, optimizing S3 the first optimized frequency range by determining a value of measurement frequencies present per decade of frequency within at least a subrange of the frequency range and reducing the determined value of measurement frequencies present per decade within the at least one subrange of the frequency range by deleting at least one measurement frequency of the at least one subrange resulting in a second optimized frequency range is performed.

The method for adapting operation settings for a frequency sweep measurement of a polymer product method refers to special operation settings for the frequency sweep measurement that leads to significant reduction of measurement time while the method accuracy remains at an adequate level for the needs of plant control via fast frequency sweep. The temperature is the same for both standard and special operation settings.

According to an exemplary embodiment of the present invention, for polyethylene, the test temperature is set to, for example, 190° C. which is the same as for the MFI measurement.

According to an exemplary embodiment of the present invention, two method parameters which contribute in reducing the measurement time have been varied:
i. Frequency range (or equivalent shear rate)—Low frequency measurements are the slowest (measurement point time is proportional to inverse frequency). Removing the low frequency measurements, leads to significant time reduction. The frequency range is narrowed down from, for example, 628-0.01 rad/s to 250-0.1 rad/s.
ii. Number of measurement points per decade—This corresponds to the amount of measurement points measured per ‘decade’ (referring to the logarithmic scale) within the intended frequency range. This parameter has been changed from for example, 5 to 3 point/decade.

According to an exemplary embodiment of the present invention, settings lead to significant reduction of the measurement time. Indicatively, the frequency sweep sample preparation and measurement time varies between 60 to 120 minutes, depending on the sample rheological characteristics, i.e. high viscosity polymers require higher measurement times. The use of the above settings reduces the measurement time to a range of 10 to 15 minutes.

The method for adapting operation settings for a frequency sweep measurement of a polymer product refers offers an attractive method for use in the plant environment:

The Fast frequency sweep is able to provide the experimental results faster. The sample preparation method is comparable to the currently employed melt index method.

The method equipment complexity and the technical competence required are at the same level with MFI method.

The method for adapting operation settings for a frequency sweep measurement of a polymer product provides a more complete rheological insight on the complex shear viscosity curve compared to MFI that corresponds to a single point of the curve (at an unknown shear rate) and the acquired complex viscosity curve is a unique fingerprint for the characterized polymer, reflecting its microstructure in terms of MWD and comonomer content.

On the contrary, MFI may be misleading since different MWD may lead to the same MFI value.

The reduced shear rate range of the method still provides adequate feedback information for the scope of plant monitoring and control.

FIG. 2 shows a schematic diagram of an apparatus for adapting operation settings for a frequency sweep measurement of a polymer product according to an exemplary embodiment of the invention.

An apparatus 100 apparatus for adapting operation settings for a frequency sweep measurement of a polymer product is provided, the apparatus 100 comprises data memory 10 and a processor 20.

The data memory 10 is configured to provide at least one set of operation setting parameters comprising at least one frequency range for the frequency sweep measurement of the polymer product, the at least one frequency range comprising a plurality of specific measurement frequencies.

The processor 20 is configured to optimize the frequency range by deleting at least one measurement frequency of the plurality of specific measurement frequencies within a lower half of the frequency range resulting in a first optimized frequency range.

The processor 20 is configured to optimize the first optimized frequency range by determining a value of measurement frequencies present per decade of frequency within at least a subrange of the frequency range and reducing the determined value of measurement frequencies present per decade within the at least one subrange of the frequency range by deleting at least one measurement frequency of the at least one subrange resulting in a second optimized frequency range.

The processor 20 may be implemented as Digital Signal Processor, DSP, in a micro-controller or in any other side-processor or as hardware circuit within an application specific integrated circuit, ASIC, CPLD or FPGA.

The processor 20 may be implemented as a Field Programmable Gate Arrays in terms of an integrated circuit that contains large numbers of identical logic cells.

The continuous need for advanced characterization methods for plant quality monitoring and control is well established. A QC method able to exceed MFI capabilities but still able to be used in plant environment will enable better plant monitoring and control level to current polymer plants.

Furthermore, a more efficient detailed kinetics, molecular properties-oriented model predictive control model has been developed. The new modelling features are able to monitor and control the polymer molecular properties and further estimate the corresponding rheological properties. Model predictive control is a powerful process tool, nevertheless, constant process feedback is needed in order to ensure the validity of the predictions.

For the new control concept, melt index values are useful but not enough-MWD or complex viscosity over large shear rate range feedback is required. This way, the limited control feedback becomes a barrier for model predictive control steps.

It has to be noted that embodiments of the invention are described with reference to different subject matters. In particular, some embodiments are described with reference to method type claims whereas other embodiments are described with reference to the device type claims.

However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. A single processor or controller or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. A method for adapting operation settings for a frequency sweep measurement of a polymer product, the method comprising the steps of:
providing at least one set of operation setting parameters comprising at least one frequency range for the frequency sweep measurement of the polymer product, the at least one frequency range comprising a plurality of specific measurement frequencies;
optimizing the frequency range by deleting at least one measurement frequency of the plurality of specific measurement frequencies within a lower half of the frequency range resulting in a first optimized frequency range; and
optimizing the first optimized frequency range by determining a value of measurement frequencies present per decade of frequency within at least a subrange of the frequency range and reducing the determined value of measurement frequencies present per decade within the at least one subrange of the frequency range by deleting at least one measurement frequency of the at least one subrange resulting in a second optimized frequency range.

2. The method according to claim 1, wherein after the step of optimizing the frequency range by deleting at least one measurement frequency, the first optimized frequency range is further optimized by equally distributing the remaining specific measurement frequencies within the lower half of the frequency range or within the complete frequency range.

3. The method according to claim 1, wherein after the step of optimizing the first optimized frequency range, the second optimized frequency range is further optimized by equally distributing the remaining specific measurement frequencies within the subrange of the frequency range or within the complete frequency range.

4. The method according to claim 1, wherein the method further comprises the step of validating a consistency of the second optimized frequency range by comparing a calculated complex viscosity value to a measured melt flow index value using the second optimized frequency range.

5. The method according to claim 1, wherein the method further comprises the steps of:
applying the second optimized frequency range for quality control measurements of the polymer product; and/or
applying the second optimized frequency range for a currently employed melt flow index measurement.

6. The method according to claim 1, wherein the method further comprises the step of generating the second optimized frequency range for a specific polymer grade of interest of the polymer product.

7. The method according to claim 6, wherein by generating the second optimized frequency range for the specific polymer grade of interest of the polymer product, a frequency sweep fingerprint for the specific polymer grade is defined.

8. The method according to claim 1, wherein the set of operation setting parameters comprises at least one shear rate.

9. An apparatus for adapting operation settings for a frequency sweep measurement of a polymer product, the apparatus comprising
a data memory which is configured to provide at least one set of operation setting parameters comprising at least one frequency range for the frequency sweep measurement of the polymer product, the at least one frequency range comprising a plurality of specific measurement frequencies; and
a processor, which is configured to optimize the frequency range by deleting at least one measurement frequency of the plurality of specific measurement frequencies within a lower half of the frequency range resulting in a first optimized frequency range;
wherein the processor is further configured to optimize the first optimized frequency range by determining a value of measurement frequencies present per decade of frequency within at least a subrange of the frequency range and reducing the determined value of measurement frequencies present per decade within the at least one subrange of the frequency range by deleting at least one measurement frequency of the at least one subrange resulting in a second optimized frequency range.

10. The apparatus according to claim 9, wherein the processor is further configured to optimize the first optimized frequency range by equally distributing the remaining specific measurement frequencies within the lower half of the frequency range or within the complete frequency range.

11. The apparatus according to claim 9, wherein the processor is further configured to optimize the second optimized frequency range by equally distributing the remaining specific measurement frequencies within the subrange of the frequency range or within the complete frequency range.

12. The apparatus according to claim 9, wherein the processor is further configured to validate a consistency of the second optimized frequency range by comparing a calculated complex viscosity value to a measured melt flow index value using the second optimized frequency range.

13. The apparatus according to claim 9, wherein the processor is further configured to generate the second optimized frequency range for a specific polymer grade of interest of the polymer product.

14. The apparatus according to claim 9, wherein the processor is further configured apply the second optimized frequency range for quality control measurements of the polymer product; and/or
wherein the processor is further configured to apply the second optimized frequency range for a currently employed melt flow index measurement.

15. A computer-readable medium comprising instructions which, when executed by a computer, cause the computer to carry out the steps of the method of claim 1.
